(19) United States
(12) Plant Patent Application Publication
(10) Pub. No.: US 2006/0123521 P1
(43) Pub. Date: Jun. 8, 2006

(54) SUTERA PLANT NAMED 'SUTCATRABL'

(50) SUTERA DIFFUSA; SUTERA

(75) Inventor: Henricus G.W. Stemkens, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/002,472

(22) Filed: Dec. 2, 2004

(51) Int. Cl. A01H 5/00

(52) U.S. Cl. PLT/263

(57) ABSTRACT

A new and distinct variety of Sutera plant particularly distinguished by its very big blue flowers, early to flower, thick stems and a growing habit that is spreading and trailing.

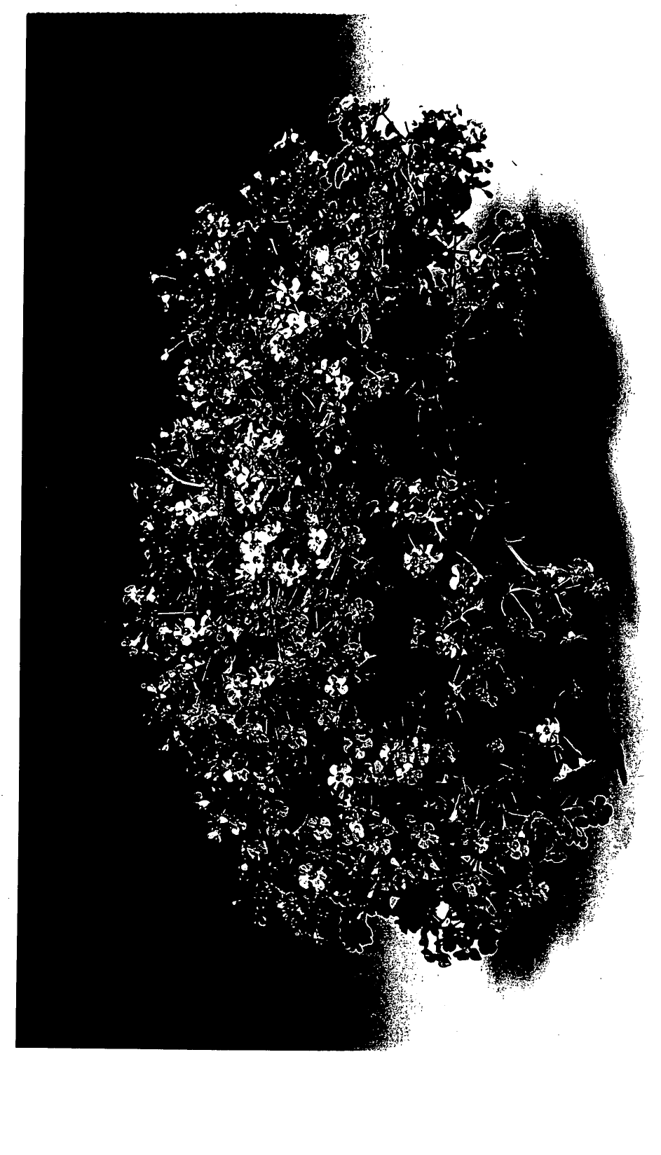

### LATIN NAME OF THE GENUS AND SPECIES OF THE PLANT CLAIMED

Sutera diffusa

### VARIETAL DENOMINATION

‘Sutcatrabl’

### BACKGROUND OF THE NEW PLANT

The present invention comprises a new distinct cultivar of Sutera, botanically known as Sutera diffusa.

The new cultivar is propagated from cuttings resulting from the cross of ‘B21’ and ‘B23’. ‘B21’ is a lavender blue flowering Sutera having a spreading, trailing habit. ‘B21’ is not commercially available and is not known by any synonyms. ‘B23’ is a white flowering Sutera having a larger than normal flower. ‘B23’ is not commercially available and is not known by any synonyms. Neither ‘B21’ or ‘B23’ has been patented.

As a result of this cross the present cultivar was created in 2000 in Enkhuizen, Netherlands and has been repeatedly asexually reproduced by cuttings in Enkhuizen, Netherlands and Sarrians, France over a three year period. It has been found to retain its distinctive characteristics through successive propagations, and this novelty appears to be firmly fixed.

This new Sutera plant is an annual in most climatical zones in the US, only in zones 9 and 10 it is a perennial plant.

‘Sutcatrabl’ is coming from the same cross as the variety ‘Sutharis’, Plant Pat. 15,254, from which it differs in having a dark blue color instead of a lavender color and having smaller leaves.

Also ‘Sutcabl’ is from the same cross, and is applied for Plant Patent at the same time. ‘Sutcabl’ has a lighter flower color, smaller flower size and a more compact habit than ‘Sutcatrabl.’

### SUMMARY OF THE INVENTION

It was found that the cultivar of the present invention:
a) Exhibits big, dark blue flowers;
b) has thick stems; and
c) exhibits a spreading and trailing growth habit

### DESCRIPTION OF THE DRAWING

This new Sutera plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Sutera. The data that define these characteristics were collected from asexual reproductions carried out in Enkhuizen, Netherlands. The plant history was taken on 15 weeks old plants, blossomed under natural light in a greenhouse and grown in a 11 cm container. Colour readings were taken in the greenhouse under ambient light. Colour references are primarily to the RHS Colour Chart of The Royal Horticultural Society of London.

TABLE 1
Differences between the new cultivar ‘Sutcatrabl’, its
parents and a similar cultivar

| | ‘Sutcatrabl’ | ‘B21’ | ‘B23’ | ‘Blue Showers’ |
|---|---|---|---|---|
| Flower color | Deep Blue | Deep blue | White | Blue |
| Stem thickness | Thick | Medium | Thick | Thin |
| Plant habit | Trailing | Trailing | Compact | Trailing |
| Flower size | Big | Big | Small | Small |

The commercial name of the most resembling variety is ‘Blue Showers’. The patented name of this variety is ‘Bacoble’ and its U.S. Plant Pat. No. is PP 13,181.

The Plant

| | | |
|---|---|---|
| Classification | Botanical | Sutera diffusa |
| Parentage | Female parent | A seedling named ‘B21’ is one |
| | | of our seedlings from |
| | | our B-generation of plants |
| | | bred in 1999. |
| | Pollen parent | A seedling named ‘B23’ is one |
| | | of our seedings from our B- |
| | | generation of plants bred in 1999. |

| | |
|---|---|
| Growth habit | Compact |
| Plant height | 12-17 cm |
| Spreading area of plant | 30-50 cm |
| Growth rate | Rapid growing |
| Strength | Good |
| Branching habit | Short internodes and on each |
| | knot more than two |
| | apprearing shoots give a plant |
| | that is more branched than |
| | normal |
| Blooming period | From May until November |

The Stem

| | |
|---|---|
| Diameter | 2-3 mm |
| Shape | Cylindrical |
| Anthocyanin pigmentation | Absent |
| Color of the stem | 135C |
| Length of internode | 12-30 mm, depending on the |
| | light where the plant is |
| | propagated |
| Pubescence | Sparse |

The Foliage

| Phyllotaxis | | Opposite |
|---|---|---|
| Shape of blade | | Non-symmetrical broadly ovate |
| Shape of the blade apex | | Acute |
| Shape of the blade base | | Non-symmetrical attenuate |
| Leaf margin | | Dentate |
| Venation | | Pinnate |
| Texture | Upper surface | Smooth, glabrous |
| | Lower surface | Slightly pubescent |
| Length | | 8-22 mm |
| Width | | 10-23 mm |
| Thickness | | 1-2 mm |
| Depth of incision | | Less than 1 mm |
| Color | Upper side | 135B |
| | Lower side | 137C |

| | |
|---|---|
| Length of petiole | 1 mm |
| Diameter of petiole | 1 mm |
| Color of petiole | 135B |
| Fragrance | Musk |

The Bud

| Pedicel length | | 12-16 mm |
|---|---|---|
| Pedicel diameter | | 1-2 mm |
| Pedicel color | | 135B |
| Pedicel angle | | About 60 degrees from the stem |
| Bud size | Diameter | 2 mm |
| | Length | 4-8 mm |
| Bud shape | | Obovate |
| Bud color | | 85A |
| Sepals | Color | 137C to 137B |
| | Number | 5 |
| | Shape | Erect calyx |
| | Size | 1 mm |

The Flower

| Flower diameter | | 14-20 mm |
|---|---|---|
| Flower tube length | | 6-10 mm |
| Throat diameter, distal end | | 2.5-4 mm |
| Tube diameter, proximal end | | 1.5-2.5 mm |
| Form | | Salverform |
| Symmetry | | Radial symmetric |
| Type | | Solitary, axillary |
| Color | Upper surface | 90C |
| | Lower surface | From 91C in the center to |
| | | N88C on the edge |
| Corolla tube | | Cylindrical |
| Corolla tube color | | 17D |
| Overlapping of petals | | Separate |
| No. of petals | | 5 |
| Shape of the petals | | Gamopetalous, grown together at |
| | | the base of the petal, |
| | | forming a corolla |
| Petal lobe | Length | 5-8 mm |
| | Width | 3-5 mm |
| | Shape | Spatulate |
| | Apex | Rounded |
| | Margin | Entire |
| | Texture | Smooth |
| Shape of sepals | | Gamopetalous, grown together at |
| | | the base of the sepal |
| Calyx | Length | 6-8 mm |
| | Diameter | 3 mm |
| | Shape | Narrow, linear |
| | Apex | Acute |
| | Margin | Entire |

Texture of the calyx

| | |
|---|---|
| Upper surface | Pubescent |
| Lower surface | Pubescent |

Color of the calyx

| | |
|---|---|
| Upper surface | 135C |
| Lower surface | 135C |

| | |
|---|---|
| Anthocyanin pigmentation of calyx | |
| limb | Absent |
| Flower fragrance | No fragrance detected |
| Lastingness of one flower | Flowers stay open 8-10 days |

Reproductive organs

| | |
|---|---|
| Number of reproductive organs | 1 pistil, 4 stamens |
| Pollen production | Very low |
| Stigma color | 138D |
| Stamen color | 13B |
| Stamen arrangement | Didynamous |
| Stamen type | Basifixed |
| Dehiscence of stamens | Extrorse |
| Ovary position | Ovary superior, flower hypogynous |
| Style shape | Style terete |
| Style length | 5 mm |
| Pistil length | 8 mm |

Seeds

No seeds are formed.

Roots

| | |
|---|---|
| Type of roots | Fibrous and fine, color 155A |

Physiological and ecological characteristics: No exceptional physiological and ecological differences with other cultivars of Sutera diffusa are observed.
Propagation: Propagation is done by axillary cuttings. Tips of stems are used, which take 6 days to initiate roots and 12 days to develop roots.

What is claimed is:

1. A new and distinct variety of Sutera plant named ‘Sutcatrabl’, substantially as illustrated and described herein.
